(19) United States
(12) Patent Application Publication
(10) Pub. No.: US 2007/0190620 A1
(43) Pub. Date: Aug. 16, 2007

(54) Saccharification processes

(75) Inventor: Marlene Mueller, Raleigh, NC (US)

Correspondence Address: NOVOZYMES NORTH AMERICA, INC., 500 FIFTH AVENUE, SUITE 1600, NEW YORK, NY, 10110, US

(73) Assignee: Novozymes North America, Inc., Franklinton, NC (US)

(21) Appl. No.: 10/565,514

(22) PCT Filed: Jul. 19, 2004
(86) PCT No.: PCT/US04/23127

§ 371 Date: Jan. 4, 2007

Related U.S. Application Data
(60) Provisional application No. 60/489,660, filed on Jul. 23, 2003.

(51) Int. Cl. C12P 7/06; C12P 1/02

(52) U.S. Cl. 435/105; 435/171; 435/161

(57) ABSTRACT

The present invention provides improved process of enzymatic saccharification or pre-saccharification of liquefied starch-containing material, by treating said liquefied material with glucoamylase activity at a pH from 5.5 to 6.2.

### FIELD OF THE INVENTION

The present invention relates to saccharification or pre-saccharification processes and processes for producing ethanol comprising a saccharification or pre-saccharification process step of the invention.

### BACKGROUND OF THE INVENTION

Saccharification is a well know step in starch degradation processes and is usually included in processes for producing fermentation products such as ethanol. The production of ethanol for fuel, beverages and industrial use is a major industry. Ethanol has widespread application, including the use as a gasoline additive or as a straight liquid fuel. Ethanol manufactures typically use whole grains as the starch-containing raw material. In this context enzymes are used for degradation of the starch-containing raw material into lower carbohydrates, such as sugars. Said lower carbohydrates are then fermented to produce ethanol or another fermentation product. A number of improved processes and improved enzymes for saccharification and/or fermentation have been developed and suggested in the art.

WO 99/28448 (From Novozymes A/S) concerns a thermostable glucoamylase derived from Talaromyces emersonli suitable for the saccharification step in starch conversion processes.

There is a desire to improve the saccharification process step in, for instance starch conversion processes and ethanol production processes.

### SUMMARY OF THE INVENTION

The present invention provides improved saccharification processes, in particular saccharification processes which may used as the saccharification step in processes for producing ethanol.

In the first aspect the invention relates to a process of enzymatic saccharification or pre-saccharification, wherein liquefied starch-containing material is treated with glucoamylase activity at a pH from 5.5 to 6.2 and at a temperature of 50 to 80° C. for 0.5 to 36 hours.

The saccharification or pre-saccharification step of the invention may, in a preferred embodiment, be followed by a fermentation step, for producing a fermentation product, especially ethanol.

In another aspect the invention relates to a process of producing ethanol, comprising the steps of:

a) liquefying starch-containing material,

b) saccharifying the liquefied starch-containing material obtained in step a) with a glucoamylase at a pH in the range of 5.2 to 6.2 and at a temperature of 50 to 80° C. for 0.5 to 36 hours.

c) fermenting the saccharified starch-containing material obtained in step b),

d) recovering ethanol from step c).

In a final aspect the invention relates to a process of producing ethanol, comprising the steps of:

a) liquefying starch-containing material,

b) pre-saccharifying the liquefied starch-containing material obtained in step a) with a glucoamylase at a pH in the range of 5.2 to 6.2 and at a temperature of 50 to 80° C. for 0.5 to 2 hours.

c) fermenting and saccharifying the pre-saccharified starch-containing material obtained in step b),

d) recovering ethanol from step c).

The saccharification or pre-saccharification may in a preferred embodiment be carried out in accordance with a saccharification or pre-saccharification process of the present invention.

### DETAILED DESCRIPTION OF THE INVENTION

The present invention relates to improvement of saccharification or pre-saccharification processes and processes comprising such process step, especially processes for producing fermentation products, such as especially ethanol.

In context on the present invention the term “saccharification step”, “saccharification process” or “saccharification process step” means a process of hydrolyzing oligosaccharides resulting from liquefaction of starch-containing material to especially monosaccharide sugars, such as dextrose.

The liquefied starch-containing material to be saccharified or pre-saccharified according to a process of the present invention may be any liquefied starch-containing material. Examples of starch-containing materials include tubers, roots, whole grains, corns, cobs, wheat, barley, rye, milo or cereals, sugar-containing raw materials, such as molasses, fruit materials, sugar, cane or sugar beet, potatoes, and cellulose-containing materials, such as wood or plant residues. In a preferred embodiment the starch-containing material to be liquefied is whole grains, especially whole grains selected from the group consisting of com, milo, wheat, and barley.

The starch-containing material may in a preferred embodiment be wet-milled or dry milled whole grains, especially of corn.

In wet milling, whole grains are first steeped to loosen the outer fiber and then the grains are separated into the different fractions (starch, germ, fiber, oil and protein). In dry milling, whole grains are ground in substantially dry state, to provide a meal, that is, without steeping the kernels to separate the kernels into the major components.

The milled starch-containing material may then be liquefied using any liquefaction process known in the art. During liquefaction the long chained starch is degraded into oligosaccharides. The liquefaction process may be performed by enzymatic or acid hydrolysis. Enzymatic liquefaction is typically preformed by treating the milled starch-containing material with an effective amount of alpha-amylase at a pH from 5.5 to 6.2 and at a temperature of 95-160° C., such as about 105-110° C., for a period of about 5 to 10 minutes followed by a lower temperature (about 95° C.) holding period of about 1 to 2 hours.

The liquefied starch-containing material is then saccharified or pre-saccharified, so that the oligosaccharides resulting from liquefaction step is converted by hydrolysis to monosaccharide sugars, such as dextrose. The saccharification process may be carried out in one step, i.e., until the saccharification is complete, or as a step of partial saccharification (i.e., pre-saccharification) followed by a simultaneous fermentation and saccharification step (SSF) wherein the saccharification of the oligosaccharides is completed. The saccharification is preferably performed enzymatically by addition of glucoamylase, alone or in combination with other enzymes, such as an alpha-glucosidase and/or an acid alpha-amylase, especially acid fungal alpha-amylase. A one step complete saccharification process lasts up to about 72 hours, and is performed at a pH between 3.0 and 5.5, preferably at a pH between 4.0 and 4.5, and at a temperature in the range of from about 30 to 65° C. Saccharification may also be carried out as a pre-saccharification step, lasting for about 30 to 480 minutes (i.e., 0.5 to 8 hours), preferably 60 to 180 minutes (1 to 3 hours), followed by completion of saccharification during a later fermentation step. Before initiation of the saccharification step the pH of the product resulting from the liquefaction step (referred to as “mash”) is decreased from the higher pH of the liquefaction step, i.e., from between 5.5 to 6.2, to a lower pH of between 3.0 and below 5.5, typically to a pH between 4.0 and 4.5. This pH decrease is obtained by addition of (an) acid(s), such as diluted or concentrated sulfuric acid. The pH adjustment is at least partly made in order to optimize the pH conditions for the glucoamylase(s) during saccharification. However, addition of an acid such as sulfuric acid has a number of disadvantages, including reduced lifetime of the process equipment due to the acidic conditions; the cost of acid increases the cost of the process; and addition of sulfuric acid requires increased use of filters for removing exhaust sulfur containing gases, such as SO₂ gas.

The present inventors have found that the pH conditions normally present after liquefaction of starch-containing material may surprisingly be used during saccharification or pre-saccharification without significantly reducing the resulting glucose concentration. The inventors have found that no or only a minor adjustment of the pH within the range of 5.5 to 6.2, i.e., used during liquefaction, is necessary. Therefore, the addition of acids, such as sulfuric acid, or other pH adjusting agents is not required or may at least be reduced. This is advantageous for all starch conversion processes and especially processes wherein a step is followed by a fermentation step or processes wherein a pre-saccharification (SSF). It is believed that at least part of the reason for this is that the pH conditions during liquefaction (i.e., a pH between 5.5 and 6.2) is more optimal for yeast used for fermentations to produce fermentation products, such as especially ethanol, than the pH conditions used during traditional saccharification and pre-saccharification (i.e., a pH below 5.5, preferably between 4.0 to 4.5).

In the first aspect the present invention relates to a process of enzymatic saccharification or pre-saccharification, wherein liquefied starch-containing material is treated with glucoamylase activity at a pH from 5.5 to 6.2 and at a temperature of 50 to 80° C. for 0.5 to 36 hours.

In one embodiment the saccharification process of the invention is performed at a pH between above 5.5 and 6.2, preferably between pH 5.6 and 6.0, especially about pH 5.7. In one embodiment the temperature during saccharification is kept between 60 and 70° C. In one embodiment the saccharification is carried out for between 0.5 to 24 hours, such as I to 16 hours, such as 1 to 8 hours. If the saccharification is carried out as a pre-saccharification step it may in one embodiment be carried out for a period of 0.5 to 8 hours. If the starch-containing starting material which is liquefied is prepared by dry milling, the saccharification may be carried out for between 1 and 16 hours, preferably I to 8 hours. If the starch-containing starting material is prepared by wet milling the saccharification is carried out for between 5 and 30 hours, preferably 8 to 16 hours.

The starch-containing material may be any starch-containing material, e.g., the materials exemplified above. However, preferred starch-containing materials are whole grains, preferably corn, wheat, milo or barley. In one embodiment the starch-containing material is liquefied using an alpha-amylase. Preferred are alpha-amylases of fungal or bacterial origin. More preferably, the alpha-amylase is a Bacillus alpha-amylase, such as, derived from a strain of B. licheniformis, B. amyloliquefaciens, B. subtilis and B. stearothermophilus. Other alpha-amylases include alpha-amylase derived from a strain of the Bacillus sp. NCIB 12289, NCIB 12512, NCIB 12513 or DSM 9375, all of which are described in detail in WO 95/26397, and the alpha-amylase described by Tsukamoto et al., Biochemical and Biophysical Research Communications, 151 (1988), pp. 25-31.

Contemplated variants and hybrids are described in WO 96/23874, WO 97/41213, and WO 99/19467, and include Bacillus stearothermophilus alpha-amylase (BSG alpha-amylase) variants having a double deletion corresponding to delta(181-182) compared to the wild-type BSG alpha-amylase amino acid sequence set forth in SEQ ID NO:3 disclosed in WO 99/19467 (hereby incorporated by reference). Even more preferred are Bacillus alpha-amylases, especially Bacillus stearothermophilus alpha-amylase, which have a double deletion corresponding to delta(181-182) and further comprise a N193F substitution (also denoted I181*+G182* +N193F) compared to the wild-type BSG alpha-amylase amino acid sequence set forth in SEQ ID NO:3 disclosed in WO 99/19467.

A hybrid alpha-amylase specifically contemplated comprises 445 C-terminal amino acid residues of the Bacillus licheniformis alpha-amylase (shown in SEQ ID NO: 4 of WO 99/19467) and the 37 N-terminal amino acid residues of the alpha-amylase derived from Bacillus amyloliquefaciens (shown in SEQ ID NO: 5 of WO 99/19467), with all or one or more of the following substitution: G48A+T49I+G07A+H156Y+Al81T+N190F+I201F+A209V+Q264S (using the Bacillus licheniformis numbering in SEQ ID NO: 4 of WO 99/19467). Also preferred are variants having one or more of the following mutations (or corresponding mutations in other Bacillus alpha-amylase backbones): H154Y, A181T, N190F, A209V and Q264S and/or deletion of two residues between positions 176 and 179, preferably deletion of E178 and G179 (using the SEQ ID NO: 5 numbering of WO 99/19467).

Also preferred are alpha-amylase variants derived from Bacillus amyloliquefaciens and having at least 50% homology, such as at least 60%, at least 70%, at least 80%, or even 90% homology to the sequence set forth in SEQ ID NO: 5 in WO 99/19467.

Other contemplated bacterial alpha-amylases include KSM-K36 alpha-amylase disclosed in EP 1,022,334 and deposited as FERM BP 6945 and KSM-K38 alpha-amylase disclosed in EP 1,022,334, and deposited as FERM BP-6946. Also variants therefore are contemplated, in particular the variants disclosed in WO 02/31124 (from Novozymes A/S).

Other alpha-amylase includes alpha-amylases derived from a strain of the genus Aspergillus, such as Aspergillus oryzae, Aspergillus niger and Aspergillus awamori alpha -amylases. In a preferred embodiment, the alpha-amylase is an acid alpha-amylase. In a more preferred embodiment the acid alpha-amylase is an acid fungal alpha-amylase or an acid bacterial alpha-amylase. More preferably, the acid alpha-amylase is an acid fungal alpha-amylase derived from the genus Aspergillus. A commercially available acid fungal amylase is SP288 (available from Novozymes A/S, Denmark). The term “acid alpha -amylase” means an alpha-amylase (E.C. 3.2.1.1) which added in an effective amount has optimal activity at a pH in the range of 3.0 to 7.0, preferably from 3.5 to 6.0, or more preferably from 4.0-5.0. A preferred acid fungal alpha-amylase is a Fungamyl-like alpha -amylase. In the present disclosure, the term “Fungamyl-like alpha-amylase” indicates an alpha-amylase which exhibits a high identity, i.e., more than 50%, 55%, 60%, 65%, 70%, 75%, 80%, 85% or even 90% identity to the amino acid sequence shown in SEQ ID No. 10 in WO 96/23874. Preferably the acid alpha-amylase is derived from the genus Aspergillus, preferably of the species Aspergillus niger. In a more preferred embodiment the acid fungal alpha-amylase is the one from A. niger disclosed as “AMYA_ASPNG” in the Swiss-prot/TeEMBL database under the primary accession no. P56271. Also variants of said acid fungal alpha-amylase having at least 70% identity, such as at least 80% or even at least 90% identity thereto are contemplated.

A preferred bacterial acid alpha-amylase may be derived from a strain of B. licheniformis, B. amyloliquefaciens, and B. stearothermophilus.

Suitable commercial alpha-amylase compositions include MYCOLASE™ from DSM (Gist Brochades); BAN™, TERMAMYL™ SC, FUNGAMYL™, LIQUOZYME™ X and SAN™ SUPER, SAN™ EXTRA L (from Novozymes A/S, Denmark); CLARASE™ L-40,000, DEX-LO™, SPEYME™ FRED, SPEZYME™ AA, and SPEZYME™ DELTA AA (from Genencor Int., USA). Preferred is the acid fungal alpha-amylase sold under the trade name SP 288 (available from Novozymes A/S, Denmark).

The alpha-amylase may also be a maltogenic alpha-amylase. A “maltogenic alpha-amylase ” (glucan 1,4-alpha-maltohydrolase, E.C. 3.2.1.133) is able to hydrolyze amylose and amylopectin to maltose in the alpha-configuration. A maltogenic alpha-amylase from B. stearothermophilus strain NCIB 11837 is commercially available from Novozymes A/S under the tradename NOVAMYL™. Maltogenic alpha-amylases are described in U.S. Pat. Nos. 4,598,048, 4,604,355 and 6,162,628, which are hereby incorporated by reference. Preferably, the maltogenic alpha-amylase is used in a raw starch hydrolysis process, as described, e.g., in WO 95/10627, which is hereby incorporated by reference.

The alpha-amylase may be added in effective amounts well-known in the art. When measured in AAU units the acid alpha-amylase activity is preferably present in an amount of 0.005-500 AAU/g DS, in an amount of 0.500-50 AAU/g DS, or more preferably in an amount of 0.1-10 AAU/g of DS, such as 0.5-1 AAU/g DS.

Fungal acid alpha-amylase may preferably be added in an amount of 0.010-1 AFAU/g DS, preferably from 0.1 to 0.3 AFAU/g DS, preferably around 0.2 AFAU/g DS.

The glucoamylase used according to the saccharification or pre-saccharification process of the invention may be derived from any suitable source, e.g., derived from a microorganism, such as a fungal organism, in particular a filamentous fungus; or a bacteria; or a plant.

Preferred filamentous glucoamylases include Talaromyces glucoamylases, in particular, derived from Talaromyces emersonli (WO 99/28448), Talaromyces leycettanus (U.S. Pat. No. Re. 32,153), Talaromyces duponti, Talaromyces thermophilus (U.S. Pat. No. 4,587,215). Bacterial glucoamylases contemplated include glucoamylases from the genus Clostridium, in particular C. thermoamylolyticum (EP 135,138), and C. thermohydrosulfuricum (WO 86/01831). Other preferred glucoamylases are of fungal or bacterial origin, selected from the group consisting of Aspergillus glucoamylases, in particular A. niger G1 or G2 glucoamylase (Boel et al. (1984), EMBO J. 3 (5), p. 1097-1102), or variants thereof, such as disclosed in WO 92/00381 and WO 00/04136; the A. awamoriglucoamylase (WO 84/02921), A. oryzae (Agric. Biol. Chem. (1991), 55 (4), p. 941-949), or variants or fragments thereof.

Other Aspergillus glucoamylase variants include variants to enhance the thermal stability: G137A and G139A (Chen et al. (1996), Prot Engng. 9, 499-505); D257E and D293E/Q (Chen et al. (1995), Prot. Engng. 8, 575-582); N182 (Chen et al. (1994), Biochem. J. 301, 275281); disulphide bonds, A246C (Fierobe et al. (1996), Biochemistry, 35, 8698-8704; and introduction of Pro residues in position A435 and S436 (Li et al. (1997), Protein Engng. 10, 1199-1204.

Commercially available compositions comprising glucoamylase include AMG 200L; AMG 300 L; SAN™ SUPER, SAN™ EXTRA L, SPIRIZYME™ PLUS, SPIRIZYME™ FUEL and AMG™ E (from Novozymes A/S); OPTIDEX™ 300 (from Genencor Int.); AMIGASE™ and AMIGASE™ PLUS (from DSM); G-ZYME™ G900, G-ZYME™ and G990 ZR (from Genencor Int.). The glucoamylase may in an effective amount, preferably from 0.01 to 0.6 AGU/g DS, preferably between 0.1 to 0.3 AGU/g DS.

In a preferred embodiment the saccharification or pre-saccharification process of the invention is carried out in the presence of an acid alpha-amylase, preferably an acid fungal alpha-amylase. Said alpha-amylase may be any of the ones mentioned above in context of alpha-amylases for liquefaction. As mentioned above the saccharification or pre-saccharification may advantageously be followed by a fermentation step, especially for the production of ethanol.

In one aspect the invention relates to a process of producing ethanol, comprising the steps of:
a) liquefying starch-containing material,
b) saccharifying the liquefied starch-containing material obtained in step a) with a glucoamylase at a pH in the range of 5.2 to 6.2 and at a temperature of 50 to 80° C. for 1 to 36 hours.
c) fermenting the saccharified starch-containing material obtained in step b),
d) recovering ethanol from step c).

The liquefaction step a) may according to this aspect of the invention be carried out as typically done in the art as described above. The saccharification step b) may in a preferred embodiment be a saccharification process of the invention as defined herein. In a preferred embodiment the pH during saccharification is between 5.5 and 6.2, more preferably between pH 5.6 and 6.0, especially around about pH 5.7. In a preferred embodiment the saccharification is carried out at temperature between 60 and 70° C.

The fermentation step may be carried out using conditions typically used in the art as will be described further below.

In the case that the ethanol production process includes a pre-saccharification step it may comprise the following steps:
a) liquefying starch-containing material,
b) pre-saccharifying the liquefied starch-containing material obtained in step a) with a glucoamylase at a pH in the range of 5.2 to 6.2 and at a temperature of 50 to 80° C. for 0.5 to 8 hours.
c) fermenting and saccharifying the pre-saccharified starch-containing material obtained in step b),
d) recovering ethanol from step c).

The liquefaction step a) may according to this aspect of the invention be carried out as typically done in the art such as described above. The pre-saccharification step b) may in a preferred embodiment be a pre-saccharification process of the invention as defined herein. The pre-saccharification in step b) may in a preferred embodiment be carried out at a pH between 5.5 and 6.2, more preferably between pH 5.6 and 6.0, especially about pH 5.7. In a preferred embodiment the saccharification is carried out at temperature between 60 and 70° C. The fermentation step may be carried out using conditions typically used in the art as will be described further below. According to the invention the fermentation step or SSF step may be carried out for about 24 to 96 hours, such as typically 3560 hours. In preferred embodiments, the temperature is generally between 26-34° C., in particular about 32° C., and the pH is generally from pH 36, preferably around pH 4-5. Yeast cells are preferably applied in amounts of 10⁵ to 10¹², preferably from 10⁷ to 10¹⁰, especially 5×10⁷ viable yeast count per ml of fermentation broth. During the ethanol producing phase the yeast cell count should preferably be in the range from 10⁷ to 10¹⁰, especially around 2 ×10^{8.} Further guidance in respect of using yeast for fermentation can be found in, e.g., “The Alcohol Textbook” (Editors K. Jacques, T. P. Lyons and D. R. Kelsall, Nottingham University Press, United Kingdom 1999), which is hereby incorporated by reference.

At the initiation of or during fermentation, yeast is added to the mash to ferment sugars to ethanol and carbon dioxide. Preferred yeast includes strains of Saccharomyces spp., more preferably, from Saccharomyces cerevisiae. Commercially available yeast include, e.g., Red Star®/Lesaffre Ethanol Red (available from Red Star/Lesaffre, USA), SUPERSTART (available from Alltech), GERT STRAND (available from Gert Strand AB, Sweden) and FERMIOL (available from DSM Specialties).

Ethanol may in a preferred embodiment of the invention be recovered in step d) by distillation which is a process of separating ethanol from the fermented mash, preferably, by evaporation. The vapors are preferably driven off by applying direct heat to the fermented mash. The vapors are collected, condensed and recovered as a liquid and may be redistilled to increase the ethanol concentration. Because ethanol has a higher vapor pressure than water, the vaporization of water and ethanol results in a liquid higher in ethanol. Through condensation, a highly concentrate distillate Is obtained. Normal distillation results in a liquid with a purity of about 95% vol % ethanol (190 proof). For fuel ethanol, the final proof must approach 200. To accomplish this result, the ethanol is subjected to further dehydration methods.

Various modifications of the invention described herein will become apparent to those skilled in the art. Such modifications are intended to fall within the scope of the appending claims.

### MATERIALS AND METHODS

Materials:

Talaromyces emersonii glucoamylase produced as disclosed in Example 9 of U.S. Pat. No. 6,255,084 and available from Novozymes A/S, Denmark

Aspergillus niger glucoamylase comprising Aspergillus niger acid fungal alpha-amylase in a ratio of 1:0.2 calculated based on activity units (AGU:AFAU). Available from Novozymes A/S, Denmark.

Bacterial alpha-amylase A: Bacillus stearothermophilus alpha-amylase variant with the mutations: I181*+G182*+N193F disclosed in U.S. Pat. No. 6,187,576 and available on request from Novozymes A/S, Denmark.

Methods:

Alpha-Amylase Activity (KNU)

The amylolytic activity may be determined using potato starch as substrate. This method is based on the break-down of modified potato starch by the enzyme, and the reaction is followed by mixing samples of the starch/enzyme solution with an iodine solution. Initially, a blackish-blue color is formed, but during the break-down of the starch the blue color gets weaker and gradually turns into a reddish-brown, which is compared to a colored glass standard.

One Kilo Novo alpha amylase Unit (KNU) is defined as the amount of enzyme which, under standard conditions (i.e., at 37° C.+/−0.05; 0.0003 M Ca²⁺; and pH 5.6) dextrinizes 5260 mg starch dry substance Merck Amylum solubile.

A folder EB-SM-0009.02/01 describing this analytical method in more detail is available upon request to Novozymes A/S, Denmark, which folder is hereby included by reference.

RS Determination: Reducing Sugar Assav: PHBAH

Prepare glucose standards from a stock 2 mg/mL (10mM) solution. Dilute in Carbonate/Bicarbonate buffer to give standards of 0.1, 0.075, 0.05, 0.0375, 0.025, 0.0125 and 0.0075 mg/mL. Standards can be aliquotted and frozen at −40° C. for up to 3 weeks.

Dilute samples 1:10 in Carbonate/Bicarbonate by adding 30 microL of sample to 270 microL of buffer. Mix. Additional dilutions are made serially from the initial 1:10 dilution into Carbonate/Bicarbonate as necessary to get the test response to fall on the standard curve.

Into the wells of a 0.2 mL PCR plate (Greiner), transfer 100 microL of each sample, standard and blank. Standards are analyzed in duplicate, samples in triplicate.

Add 50 microL of PHBAH reagent to each well. Seal plate with a Mylar sealer.

Place the PCR plate in a 90° C. heat block for 10 minutes.

Retrieve plate and allow cooling to ambient temperature.

Transfer 50 microL of each sample, standard and blank to a corresponding well of a 96 well assay plate (Nunc) containing 50 microL of PHBAH buffer (1:2 dilution).

Read absorbance at 405 nm.

Determination of FAU Activity

One Fungal Alpha-Amylase Unit (FAU) is defined as the amount of enzyme, which breaks down 5.26 g starch (Merck Amylum solubile Erg. B.6, Batch 9947275) per hour based upon the following standard conditions:

| | |
|---|---|
| Substrate | Soluble starch |
| Temperature | 37° C. |
| PH | 4.7 |
| Reaction time | 7-20 minutes |

Determination of Acid Alpha-Amylase Activity (AFAU)

Acid alpha-amylase activity is measured in AFAU (Acid Fungal Alpha-amylase Units), which are determined relative to an enzyme standard.

The standard used is AMG 300 L (from Novozymes A/S, Denmark, glucoamylase wildtype Aspergillus niger G1, also disclosed in Boel et al. (1984), EMBO J. 3 (5), p. 1097-1102) and WO 92/00381). The neutral alpha-amylase activity in this AMG falls after storage at room temperature for 3 weeks from approx. 1 FAU/mL to below 0.05 FAU/mL.

The acid alpha-amylase activity in this AMG standard is determined in accordance with the following description. In this method, 1 AFAU is defined as the amount of enzyme, which degrades 5.260 mg starch dry matter per hour under standard conditions.

Iodine forms a blue complex with starch but not with its degradation products. The intensity of colour is therefore directly proportional to the concentration of starch. Amylase activity is determined using reverse colorimetry as a reduction in the concentration of starch under specified analytic conditions.

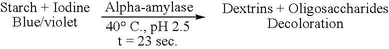

Standard Conditions/Reaction Conditions: (Per Minute)

Substrate: Starch, approx. 0.17 g/L

Buffer: Citrate, approx. 0.03 M

Iodine (I₂): 0.03 g/L

CaCl₂: 1.85 mM

pH: 2.50 ±0.05

Incubation temperature: 40° C.

Reaction time: 23 seconds

Wavelength: lambda=590 nm

Enzyme concentration: 0.025 AFAU/mL

Enzyme working range: 0.01-0.04 AFAU/mL
If further details are preferred these can be found in EB-SM-0259.02/01 available on request from Novozymes A/S, Denmark, and incorporated by reference.
   Acid Alpha-Amylase Units (AAU)

The acid alpha-amylase activity can be measured in AAU (Acid Alpha-amylase Units), which is an absolute method. One Acid Amylase Unit (AAU) is the quantity of enzyme converting 1 g of starch (100% of dry matter) per hour under standardized conditions into a product having a transmission at 620 nm after reaction with an iodine solution of known strength equal to the one of a color reference.

Standard Conditions/Reaction Conditions:

Substrate: Soluble starch. Concentration approx. 20 g DS/L.

Buffer: Citrate, approx. 0.13 M, pH=4.2

Iodine solution: 40.176 g potassium iodide +0.088 g iodine/L

City water 15°-20 dH (German degree hardness)

pH: 4.2

Incubation temperature: 30° C.

Reaction time: 11 minutes

Wavelength: 620 nm

Enzyme concentration: 0.13-0.19 AAU/mL

Enzyme working range: 0.13-0.19 AAU/mL

The starch should be Lintner starch, which is a thin-boiling starch used in the laboratory as colorimetric indicator. Lintner starch is obtained by dilute hydrochloric acid treatment of native starch so that it retains the ability to color blue with iodine. Further details can be found in EP0140410 B2, which disclosure is hereby included by reference.

Glucoamylase Activity (AGI)

Glucoamylase (equivalent to amyloglucosidase) converts starch into glucose. The amount of glucose is determined here by the glucose oxidase method for the activity determination. The method is described in the section 76-11 Starch—Glucoamylase Method with Subsequent Measurement of Glucose with Glucose Oxidase in “Approved methods of the American Association of Cereal Chemists”. Vol.1-2 AACC, from American Association of Cereal Chemists, (2000); ISBN: 1-891127-12-8.

One glucoamylase unit (AGI) is the quantity of enzyme which will form 1 micromol of glucose per minute under the standard conditions of the method.

Standard Conditions/Reaction Conditions:

Substrate: Soluble starch.

Concentration approx. 16 g dry matter/L.

Buffer: Acetate, approx. 0.04 M, pH=4.3

pH: 4.3

Incubation temperature: 60° C.

Reaction time: 15 minutes

Termination of the reaction: NaOH to a concentration of approximately 0.2 g/L (pH˜9) Enzyme concentration: 0.15-0.55 AAU/mL.

The starch should be Lintner starch, which is a thin-boiling starch used in the laboratory as colorimetric indicator. Lintner starch is obtained by dilute hydrochloric acid treatment of native starch so that it retains the ability to color blue with iodine.

Glucoamylase Activity (AGU)

The Novo Glucoamylase Unit (AGU) is defined as the amount of enzyme, which hydrolyzes 1 micromole maltose per minute under the standard conditions 37° C., pH 4.3, substrate concentration of 23.2 mM, acetate buffer 0.1 M, reaction time 5 minutes.

An autoanalyzer system may be used. Mutarotase is added to the glucose dehydrogenase reagent so that any alpha-D-glucose present is turned into beta-D-glucose. Glucose dehydrogenase reacts specifically with beta-D-glucose in the reaction mentioned above, forming NADH which is determined using a photometer at 340 nm as a measure of the original glucose concentration.

| | |
|---|---|
| AMG incubation: | |
| Substrate: | maltose 23.2 mM |
| Buffer | acetate 0.1 M |
| pH: | 4.30 ± 0.05 |
| Incubation temperature: | 37° C. ± 1 |
| Reaction time: | 5 minutes |
| Enzyme working range: | 0.5-4.0 AGU/mL |
| Color reaction: | |
| GlucDH: | 430 U/L |
| Mutarotase: | 9 U/L |
| NAD: | 0.21 mM |
| Buffer: | phosphate 0.12 M; 0.15 M NaCl |
| pH: | 7.60 ± 0.05 |
| Incubation temperature: | 37° C. ± 1 |
| Reaction time: | 5 minutes |
| Wavelength: | 340 nm |

A folder (EB-SM-0131.02/01) describing this analytical method in more detail is available on request from Novozymes A/S, Denmark, which folder is hereby included by reference.

### EXAMPLES

### Example 1

Saccharification of Liquefied Corn Mash at pH 5.7

Dry milled whole corn was liquefied using bacterial alpha-amylase A to a DE (Dextrose Equivalent) of around 12. The pH of the liquefied corn mash was adjusted to 4.5 (control) and 5.7, respectively. Saccharification was carried out in the present of 0.36 AGU/g DS Talaromyces emersonli glucoamylase produced as disclosed in Example 9 of U.S. Pat. No. 6,255,084. Another saccharification test was carried out with 0.36 AGU/g DS of Aspergillus niger glucoamylase comprising Aspergillus niger acid fungal alpha-amylase in a ratio of 1:0.2 calculated based on activity units (AGU:AFAU). Both saccharifications were carried out at 65° C. Samples were taken at T=4 and 26 hours, respectively, and analyzed using a HPLC for determination of the glucose content.

% Glucose (w/w)

| | pH 5.7 | pH 4.5 |
|---|---|---|
| 4 hours | | |
| Talaromyces emersonii | 26.39 | 26.46 |
| Glucoamylase | | |
| Aspergillus niger glucoamylase | 26.88 | 28.50 |
| with AFAU | | |
| 26 hours | | |
| Talaromyces emersonii | 32.70 | 32.70 |
| Glucoamylase | | |
| Aspergillus niger glucoamylase | 32.90 | 32.70 |
| with AFAU | | |

1-19. (canceled)

20. A process of enzymatic saccharification or pre-saccharification, wherein liquefied starch-containing material is treated with a polypeptide having glucoamylase activity at a pH from 5.5 to 6.2 and at a temperature of 50 to 80° C. for 0.5 to 36 hours.

21. The process of claim 20, wherein the saccharification is carried out at a pH between above 5.5 and 6.2.

22. The process of claim 20, wherein the saccharification is carried out at a temperature between 60 and 70° C.

23. The process of claim 20, wherein the saccharification is carried out for between 0.5 and 24 hours.

24. The process of claim 20, wherein liquefied starch material is prepared by dry milling for between 1 and 16 hours.

25. The process of claim 20, wherein liquefied starch material is prepared by wet milling for between 5 and 30 hours.

26. The process of claim 20, wherein the starch-containing material is prepared from whole grains.

27. The process of claim 20, wherein the saccharification or pre-saccharification step is followed by a fermentation step.

28. The process of claim 27, wherein the fermentation is carried out by yeast.

29. The process of claim 20, wherein the starch-containing material to be saccharified is liquefied using an alpha-amylase.

30. The process of claim 20, wherein the polypeptide is derived from a fungal organism.

31. The process of claim 30, wherein the polypeptide is derived from Aspergillus or Talaromyces.

32. The process of claim 31, wherein the polypeptide is derived from A. niger or T emersonii.

33. The process of claim 20, wherein the polypeptide is added in an amount of 0.01 to 0.6 AGU/g DS.

34. The process of claim 20, wherein the saccharification is carried out in the presence of an acid alpha-amylase, preferably an acid fungal alpha-amylase.

35. The process of claim 20, wherein the saccharification is a pre-saccharification carried out for 0.5 to 8 hours.

36. A process of producing ethanol, comprising the steps of:
a) liquefying starch-containing material,
b) saccharifying the liquefied starch-containing material obtained in step a) with a glucoamylase at a pH in the range of 5.2 to 6.2 and at a temperature of 50 to 80° C. for 1 to 36 hours.
c) fermenting the saccharified starch-containing material obtained in step b),
d) recovering ethanol from step c).

37. A process of producing ethanol, comprising the steps of:
a) liquefying starch-containing material,
b) pre-saccharifying the liquefied starch-containing material obtained in step a) with a glucoamylase at a pH in the range of 5.2 to 6.2 and at a temperature of 50 to 80° C. for 0.5 to 8 hours.
c) fermenting and saccharifying the pre-saccharified starch-containing material obtained in step b),
d) recovering ethanol from step c).
